(19) United States
(12) Patent Application Publication
(10) Pub. No.: US 2023/0069017 A1
(43) Pub. Date: Mar. 2, 2023

(54) SYSTEMS AND METHODS FOR IMAGE RECONSTRUCTION

(71) Applicant: UIH AMERICA, INC., Houston, TX (US)

(72) Inventors: Tao FENG, Houston, TX (US); Hongdi LI, Houston, TX (US)

(73) Assignee: UIH AMERICA, INC., Houston, TX (US)

(21) Appl. No.: 17/446,299

(22) Filed: Aug. 29, 2021

(51) Int. Cl. A61B 6/03; A61B 6/00; A61K 49/00

(52) U.S. Cl. CPC A61B 6/037; A61B 6/507; A61B 6/5217; A61K 49/0052

(57) ABSTRACT

The present disclosure is related to systems and methods for image reconstruction. The method may include obtaining at least one positron emission tomography (PET) image of a subject. The at least one PET image may be generated based on PET data acquired during an examination period. In the examination period, the subject may be injected with a tracer. The method may also include determining, based on the at least one PET image, an input function that reflects a concentration change of the tracer in the subject during the examination period. The method may further include generating a parametric image based on the input function and the at least one PET image according to a non-linear parametric estimation algorithm. The parametric image may reflect a kinetic parameter of the tracer in the subject.

### TECHNICAL FIELD

This disclosure generally relates to systems and methods for image reconstruction, and more particularly, relates to systems and methods for parametric imaging.

### BACKGROUND

PET technology has been widely used for clinical examination and medical diagnosis. Parametric imaging technique in PET can provide a quantitative measurement result having a higher accuracy compared with standardized uptake value (SUV) imaging technique. For example, the parametric imaging technique can provide voxel-level dynamics of a tracer uptake by applying kinetic modeling for each individual voxel. However, compared with the SUV imaging technique, the parametric imaging technique usually needs a longer scan time and a more complex protocol, which limits the clinical application of the parametric imaging technique. Thus, it is desirable to develop methods and systems for parametric imaging, thereby improving the efficiency of parametric imaging.

### SUMMARY

According to an aspect of the present disclosure, a method for image reconstruction may be provided. The method may be implemented on a computing device having at least one processor and at least one storage device. The method may include obtaining at least one positron emission tomography (PET) image of a subject. The at least one PET image may be generated based on PET data acquired during an examination period. In the examination period, the subject may be injected with a tracer. The method may also include determining, based on the at least one PET image, an input function that reflects a concentration change of the tracer in the subject during the examination period. The method may further include generating a parametric image based on the input function and the at least one PET image according to a non-linear parametric estimation algorithm. The parametric image may reflect a kinetic parameter of the tracer in the subject.

In some embodiments, the at least one PET image may include a plurality of PET images. The method may include obtaining the plurality of PET images by performing a multi-point scan on the subject. To perform the multi-point scan, the tracer may be injected into the subject at an initial time point during the examination period, and a plurality of PET scans may be performed on the subject during a plurality of scan periods after the initial time point. Each of the plurality of PET scans may be performed during one of the plurality of scan periods with a time interval between each pair of adjacent PET scans among the plurality of PET scans.

In some embodiments, the method may include obtaining a reference input function relating to the subject. The method may include, for each of the plurality of scan periods, determining a candidate input function that reflects a concentration change of the tracer in the subject during the scan period based on the PET image corresponding to the scan period. The method may include generating the input function by transforming the reference input function based on the plurality of candidate input functions.

In some embodiments, the at least one PET image may include one PET image of the subject. The method may include obtaining the PET image by performing a dual injection scan on the subject. To perform the dual injection scan on the subject, a first portion of the tracer may be injected into the subject at a first time point during the examination period and a second portion of the tracer may be injected into the subject at a second time point after the first time point during the examination period. A PET scan may be performed during a scan period. The scan period may start after the first time point and before the second time point, and the scan period may end after the second time point.

In some embodiments, the method may include obtaining a reference input function relating to the subject. The method may include determining, based on the PET image, a first candidate input function that reflects a concentration change of the tracer in the subject during the scan period. The method may include determining a second candidate input function that reflects a concentration change of the tracer in the subject during a period after the first time point based on the first candidate input function, the first portion, and the second portion. The method may include generating the input function by transforming the reference input function based on the first and second candidate input functions.

In some embodiments, the method may include generating a compartment model used to model tracer dynamics within the subject. The method may include generating the parametric image based on the compartment model, the input function, and the at least one PET image according to the non-linear parametric estimation algorithm.

In some embodiments, the compartment model may be used to model at least one of a forward transport of the tracer from the plasma of the subject to the tissue of the subject, a backward transport of the tracer from the plasma to the tissue, a phosphorylation process in the tissue of the subject, or a dephosphorylation process in the tissue of the subject.

In some embodiments, the method may include generating a relationship function between the compartment model, the input function, and the at least one PET image. The method may include generating the parametric image based on the relationship function according to the non-linear parametric estimation algorithm.

In some embodiments, the non-linear parametric estimation algorithm may include a maximum likelihood estimation (MLE) algorithm.

In some embodiments, the tracer may be an ¹⁸F-fluorodeoxyglucose (FDG).

In some embodiments, the parametric image may include a Ki image.

According to another aspect of the present disclosure, a system for image reconstruction may be provided. The system may include at least one storage device storing executable instructions, and at least one processor in communication with the at least one storage device. When executing the executable instructions, the at least one processor may cause the system to perform a method. The method may include obtaining at least one positron emission tomography (PET) image of a subject. The at least one PET image may be generated based on PET data acquired during an examination period. In the examination period, the subject may be injected with a tracer. The method may include determining, based on the at least one PET image, an input function that reflects a concentration change of the tracer in the subject during the examination period. The method may include generating a parametric image based on the input function and the at least one PET image according to a non-linear parametric estimation algorithm. The parametric image may reflect a kinetic parameter of the tracer in the subject.

According to yet another aspect of the present disclosure, a non-transitory computer readable medium may be provided. The non-transitory computer readable may include at least one set of instructions. When executed by at least one processor of a computing device, the at least one set of instructions may cause the computing device to perform a method. The method may include obtaining at least one positron emission tomography (PET) image of a subject. The at least one PET image may be generated based on PET data acquired during an examination period. In the examination period, the subject may be injected with a tracer. The method may include determining, based on the at least one PET image, an input function that reflects a concentration change of the tracer in the subject during the examination period. The method may include generating a parametric image based on the input function and the at least one PET image according to a non-linear parametric estimation algorithm. The parametric image may reflect a kinetic parameter of the tracer in the subject.

According to an aspect of the present disclosure, a method for image reconstruction may be provided. The method may be implemented on a computing device having at least one processor and at least one storage device. The method may include obtaining at least one positron emission tomography (PET) image of a subject. The at least one PET image may be generated based on PET data acquired during an examination period. In the examination period, the subject may be injected with a tracer. A multi-point scan or a dual injection scan may be performed on the subject. A total time of one or more scan periods of the multi-point scan or the dual injection scan may be less than or equal to 10 minutes. The method may include generating a parametric image based on the at least one PET image according to a non-linear parametric estimation algorithm. The parametric image may reflect a kinetic parameter of the tracer in the subject.

In some embodiments, the at least one PET image may include a plurality of PET images. The method may include obtaining the plurality of PET images by performing the multi-point scan on the subject. The tracer may be injected into the subject at an initial time point during the examination period. A plurality of PET scans may be sequentially performed on the subject during a plurality of scan periods after the initial time point. Each of the plurality of PET scans may be performed during one of the plurality of scan periods with a time interval between each pair of adjacent PET scans among the plurality of PET scans.

In some embodiments, the method may include registering the plurality of PET images.

In some embodiments, the at least one PET image may include one PET image of the subject. The method may include obtaining the PET image by performing the dual injection scan on the subject. A first portion of the tracer may be injected into the subject at a first time point during the examination period and a second portion of the tracer may be injected into the subject at a second time point after the first time point during the examination period. A PET scan may be performed during a scan period. The scan period may start after the first time point and before the second time point. The scan period may end after the second time point.

In some embodiments, the method may include determining, based on the at least one PET image, an input function that reflects a concentration change of the tracer in the subject during the examination period.

In some embodiments, the parametric image may include a Ki image.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary imaging system according to some embodiments of the present disclosure;

FIG. 2 is a schematic diagram illustrating exemplary hardware and/or software components of a computing device according to some embodiments of the present disclosure;

FIG. 3 is a schematic diagram illustrating exemplary hardware and/or software components of a mobile device according to some embodiments of the present disclosure;

FIG. 4 is a schematic diagram illustrating an exemplary processing device according to some embodiments of the present disclosure;

FIG. 5 is a flowchart illustrating an exemplary process for generating a parametric image according to some embodiments of the present disclosure;

FIG. 6 is a flowchart illustrating an exemplary process for generating a parametric image according to some embodiments of the present disclosure;

FIG. 7A is a schematic diagram illustrating an exemplary multi-point scan according to some embodiments of the present disclosure;

FIG. 7B is a schematic diagram illustrating an exemplary dual injection scan according to some embodiments of the present disclosure; and

FIG. 8 illustrates exemplary Ki images of a patient according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. Also, the term “exemplary” is intended to refer to an example or illustration.

It will be understood that the terms “system,” “engine,” “unit,” “module,” and/or “block” used herein are one method to distinguish different components, elements, parts, sections or assembly of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that, although the terms “first,” “second,” “third,” etc., may be used herein to describe various elements, these elements should not be limited by these terms. These terms are only used to distinguish one element from another. For example, a first element could be termed a second element, and, similarly, a second element could be termed a first element, without departing from the scope of exemplary embodiments of the present disclosure.

Spatial and functional relationships between elements are described using various terms, including “connected,” “attached,” and “mounted.” Unless explicitly described as being “direct,” when a relationship between first and second elements is described in the present disclosure, that relationship includes a direct relationship where no other intervening elements are present between the first and second elements, and also an indirect relationship where one or more intervening elements are present (either spatially or functionally) between the first and second elements. In contrast, when an element is referred to as being “directly” connected, attached, or positioned to another element, there are no intervening elements present. Other words used to describe the relationship between elements should be interpreted in a like fashion (e.g., “between,” versus “directly between,” “adjacent,” versus “directly adjacent,” etc.).

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The term “image” in the present disclosure is used to collectively refer to image data (e.g., scan data, projection data) and/or images of various forms, including a two-dimensional (2D) image, a three-dimensional (3D) image, a four-dimensional (4D), etc. The term “pixel” and “voxel” in the present disclosure are used interchangeably to refer to an element of an image. The term “anatomical structure” in the present disclosure may refer to gas (e.g., air), liquid (e.g., water), solid (e.g., stone), cell, tissue, organ of a subject, or any combination thereof, which may be displayed in an image and really exist in or on the subject's body. The term “region,” “location,” and “area” in the present disclosure may refer to a location of an anatomical structure shown in the image or an actual location of the anatomical structure existing in or on the subject's body, since the image may indicate the actual location of a certain anatomical structure existing in or on the subject's body.

Provided herein are systems and components for an imaging system. In some embodiments, the imaging system may include a single modality imaging system and/or a multi-modality imaging system. The single modality imaging system may include, for example, a PET system, a SPECT system, or the like, or any combination thereof. The multi-modality imaging system may include, for example, a positron emission tomography-X-ray imaging (PET-X-ray) system, a single photon emission computed tomography-magnetic resonance imaging (SPECT-MRI) system, a positron emission tomography-computed tomography (PET-CT) system, a digital subtraction angiography-magnetic resonance imaging (DSA-MRI) system, etc. It should be noted that the imaging system described below is merely provided for illustration purposes, and not intended to limit the scope of the present disclosure.

An aspect of the present disclosure relates to systems and methods for generating a parametric image. According to some embodiments of the present disclosure, a processing device may obtain at least one PET image of a subject. The at least one PET image may be generated based on PET data acquired during an examination period in which the subject is injected with a tracer. For example, the at least one PET image may be obtained by performing a multi-point scan or a dual injection scan on the subject. The multi-point scan may be implemented by sequentially performing a plurality of PET scans on the subject after all the tracer is injected into the subject at an initial time point of the examination period. The dual injection scan may be implemented by injecting the tracer into the subject via two injections and performing a single PET scan on the subject, wherein the PET scan may start between the first injection and the second injection, and end after the second injection.

The processing device may then determine an input function that reflects a concentration change of the tracer in the subject during the examination period based on the at least one PET image. For example, the input function may be determined based on an image-derived input function (also referred to as a candidate input function) and a population-based input function (also referred to as a reference input function). As used herein, an image-derived input function refers to an input function that is determined based on one or more PET images of a subject. A population-based input function refers to an input function of a subject that is determined based on a plurality of sample input functions corresponding to a plurality of sample subjects other than the subject.

The processing device may further generate a parametric image (e.g., a Ki image) based on the input function and the at least one PET image. The parametric image may reflect a kinetic parameter of the tracer in the subject. For example, the parametric image may be generated based on a relationship function between a compartment model, the input function, and the at least one PET image according to a non-linear parametric estimation algorithm (e.g., a maximum likelihood estimation algorithm).

Conventionally, a parametric image of a subject needs to be acquired by performing a continuous PET scan with a long scan time (e.g., tens of minutes), and a Patlak model may be used to determine the parametric image (e.g., a Ki image). The Patlak model is a linear model, and Ki may correspond to the slope of the linear model. However, if the imaging time is not long enough (e.g., less than 10 minutes), there may not be enough PET data for the linear model to determine the parametric image. According to some embodiments of the present disclosure, the parametric image may be generated based on PET data obtained in a relatively shorter imaging time (e.g., less than or equal to 10 minutes) according to the non-linear parametric estimation algorithm (e.g., the maximum likelihood estimation algorithm). Compared with the conventional approach (e.g., parametric imaging using a Patlak model), the systems and methods disclosed herein may be used to generate a parametric image with a relatively shorter imaging time (e.g., less than or equal to 10 minutes), which may improve the imaging efficiency and promote a clinical application of the parametric imaging.

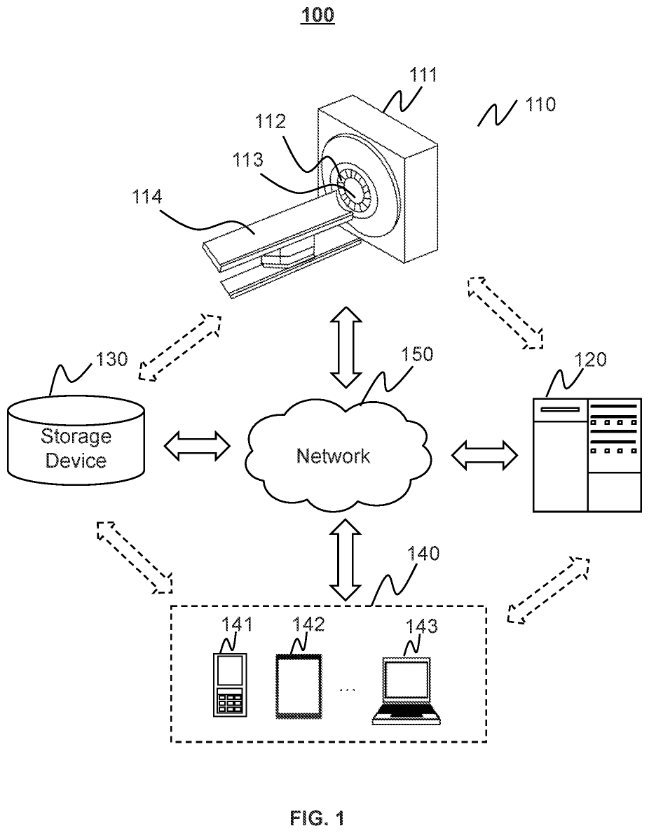

FIG. 1 is a schematic diagram illustrating an exemplary imaging system according to some embodiments of the present disclosure. As illustrated, an imaging system 100 may include an imaging device 110, a processing device 120, a storage device 130, a terminal 140, and a network 150. The components of the imaging system 100 may be connected in one or more of various ways. Merely by way of example, as illustrated in FIG. 1, the imaging device 110 may be connected to the processing device 120 directly as indicated by the bi-directional arrow in dotted lines linking the imaging device 110 and the processing device 120, or through the network 150. As another example, the storage device 130 may be connected to the imaging device 110 directly as indicated by the bi-directional arrow in dotted lines linking the imaging device 110 and the storage device 130, or through the network 150. As still another example, the terminal 140 may be connected to the processing device 120 directly as indicated by the bi-directional arrow in dotted lines linking the terminal 140 and the processing device 120, or through the network 150.

In some embodiments, the imaging device 110 may scan a subject to acquire data relating to the subject. In some embodiments, the imaging device 110 may be an emission computed tomography (ECT) device, a positron emission tomography (PET) device, a single photon emission computed tomography (SPECT) device, a multi-modality device, or the like, or any combination thereof. Exemplary multi-modality device may include a CT-PET device, an MR-PET device, or the like. In some embodiments, the multi-modality imaging device may include modules and/or components for performing PET imaging and/or related analysis.

In some embodiments, the imaging device 110 may be a PET device including a gantry 111, a detector 112, a detection region 113, and a table 114. The gantry 111 may support the detector 112. The subject may be placed on the table 114 and moved into the detection region 113 for scanning along the Z axis as illustrated in FIG. 1. The detector 112 may detect radiation events (e.g., gamma photons) emitted from the detection region 113. In some embodiments, the detector 112 may include one or more detector units. The detector 112 may include a scintillation detector (e.g., a cesium iodide detector), a gas detector, etc. The detector 112 may be and/or include a single-row detector in which a plurality of detector units are arranged in a single row and/or a multi-row detector in which a plurality of detector units are arranged in multiple rows.

The subject may be biological or non-biological. For example, the subject may include a patient, a man-made object, etc. As another example, the subject may include a specific portion, an organ, and/or tissue of the patient. Specifically, the subject may include the head, the neck, the thorax, the heart, the stomach, a blood vessel, soft tissue, a tumor, or the like, or any combination thereof. In the present disclosure, “object” and “subject” are used interchangeably.

The processing device 120 may process data and/or information obtained from the imaging device 110, the storage device 130, and/or the terminal(s) 140. For example, the processing device 120 may obtain at least one PET image of the subject. As another example, the processing device 120 may determine an input function based on at least one PET image. As still another example, the processing device 120 may generate a parametric image based on an input function and at least one PET image.

In some embodiments, the processing device 120 may be a single server or a server group. The server group may be centralized or distributed. In some embodiments, the processing device 120 may be local or remote. For example, the processing device 120 may access information and/or data from the imaging device 110, the storage device 130, and/or the terminal(s) 140 via the network 150. As another example, the processing device 120 may be directly connected to the imaging device 110, the terminal(s) 140, and/or the storage device 130 to access information and/or data. In some embodiments, the processing device 120 may be implemented on a cloud platform. For example, the cloud platform may include a private cloud, a public cloud, a hybrid cloud, a community cloud, a distributed cloud, an inter-cloud, a multi-cloud, or the like, or a combination thereof. In some embodiments, the processing device 120 may be part of the terminal 140. In some embodiments, the processing device 120 may be part of the imaging device 110.

The storage device 130 may store data, instructions, and/or any other information. In some embodiments, the storage device 130 may store data obtained from the imaging device 110, the processing device 120, and/or the terminal(s) 140. The data may include image data acquired by the processing device 120, algorithms and/or models for processing the image data, etc. For example, the storage device 130 may store a PET image of the subject obtained from a PET device (e.g., the imaging device 110). As still another example, the storage device 130 may store an input function determined by the processing device 120. As still another example, the storage device 130 may store a parametric image generated by the processing device 120.

In some embodiments, the storage device 130 may store data and/or instructions that the processing device 120 and/or the terminal 140 may execute or use to perform exemplary methods described in the present disclosure. In some embodiments, the storage device 130 may include a mass storage device, removable storage device, a volatile read-and-write memory, a read-only memory (ROM), or the like, or any combination thereof. Exemplary mass storage device may include a magnetic disk, an optical disk, a solid-state drive, etc. Exemplary removable storage device may include a flash drive, a floppy disk, an optical disk, a memory card, a zip disk, a magnetic tape, etc. Exemplary volatile read-and-write memories may include a random-access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 130 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 130 may be connected to the network 150 to communicate with one or more other components in the imaging system 100 (e.g., the processing device 120, the terminal(s) 140). One or more components in the imaging system 100 may access the data or instructions stored in the storage device 130 via the network 150. In some embodiments, the storage device 130 may be integrated into the imaging device 110.

The terminal(s) 140 may be connected to and/or communicate with the imaging device 110, the processing device 120, and/or the storage device 130. In some embodiments, the terminal 140 may include a mobile device 141, a tablet computer 142, a laptop computer 143, or the like, or any combination thereof. For example, the mobile device 141 may include a mobile phone, a personal digital assistant (PDA), a gaming device, a navigation device, a point of sale (POS) device, a laptop, a tablet computer, a desktop, or the like, or any combination thereof. In some embodiments, the terminal 140 may include an input device, an output device, etc. The input device may include alphanumeric and other keys that may be input via a keyboard, a touchscreen (for example, with haptics or tactile feedback), a speech input, an eye tracking input, a brain monitoring system, or any other comparable input mechanism. Other types of the input device may include a cursor control device, such as a mouse, a trackball, or cursor direction keys, etc. The output device may include a display, a printer, or the like, or any combination thereof.

The network 150 may include any suitable network that can facilitate the exchange of information and/or data for the imaging system 100. In some embodiments, one or more components of the imaging system 100 (e.g., the imaging device 110, the processing device 120, the storage device 130, the terminal(s) 140, etc.) may communicate information and/or data with one or more other components of the imaging system 100 via the network 150. For example, the processing device 120 and/or the terminal 140 may obtain a PET image from the imaging device 110 via the network 150. As another example, the processing device 120 and/or the terminal 140 may obtain information stored in the storage device 130 via the network 150. The network 150 may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network, etc.), a cellular network (e.g., a Long Term Evolution (LTE) network), a frame relay network, a virtual private network (VPN), a satellite network, a telephone network, routers, hubs, switches, server computers, and/or any combination thereof. For example, the network 150 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 150 may include one or more network access points. For example, the network 150 may include wired and/or wireless network access points such as base stations and/or internet exchange points through which one or more components of the Imaging system 100 may be connected to the network 150 to exchange data and/or information.

This description is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. However, those variations and modifications do not depart the scope of the present disclosure. In some embodiments, the imaging system 100 may include one or more additional components and/or one or more components of the imaging system 100 described above may be omitted. Additionally or alternatively, two or more components of the imaging system 100 may be integrated into a single component. A component of the imaging system 100 may be implemented on two or more sub-components.

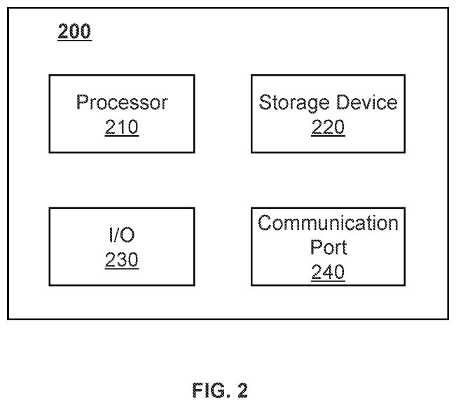

FIG. 2 is a schematic diagram illustrating exemplary hardware and/or software components of a computing device 200 according to some embodiments of the present disclosure. In some embodiments, one or more components of the imaging system 100 (e.g., the processing device 120, a terminal 140) may be implemented on the computing device 200. As illustrated in FIG. 2, the computing device 200 may include a processor 210, a storage device 220, an input/output (I/O) 230, and a communication port 240.

The processor 210 may execute computer instructions (e.g., program code) and perform functions of the processing device 120 in accordance with techniques described herein. The computer instructions may include, for example, routines, programs, objects, components, data structures, procedures, modules, and functions, which perform particular functions described herein. For example, the processor 210 may process imaging data obtained from the imaging device 110, the terminal(s) 140, the storage device 130, and/or any other component of the imaging system 100. In some embodiments, the processor 210 may include one or more hardware processors, such as a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combination thereof.

Merely for illustration, only one processor is described in the computing device 200. However, it should be noted that the computing device 200 in the present disclosure may also include multiple processors. Thus operations and/or method steps that are performed by one processor as described in the present disclosure may also be jointly or separately performed by the multiple processors. For example, if in the present disclosure the processor of the computing device 200 executes both process A and process B, it should be understood that process A and process B may also be performed by two or more different processors jointly or separately in the computing device 200 (e.g., a first processor executes process A and a second processor executes process B, or the first and second processors jointly execute processes A and B).

The storage device 220 may store data/information obtained from the imaging device 110, the terminal(s) 140, the storage device 130, and/or any other component of the Imaging system 100. The storage device 220 may be similar to the storage device 130 described in connection with FIG. 1, and the detailed descriptions are not repeated here.

The I/O 230 may input and/or output signals, data, information, etc. In some embodiments, the I/O 230 may enable a user interaction with the processing device 120. In some embodiments, the I/O 230 may include an input device and an output device. Examples of the input device may include a keyboard, a mouse, a touchscreen, a microphone, a sound recording device, or the like, or a combination thereof. Examples of the output device may include a display device, a loudspeaker, a printer, a projector, or the like, or a combination thereof. Examples of the display device may include a liquid crystal display (LCD), a light-emitting diode (LED)-based display, a flat panel display, a curved screen, a television device, a cathode ray tube (CRT), a touchscreen, or the like, or a combination thereof.

The communication port 240 may be connected to a network (e.g., the network 150) to facilitate data communications. The communication port 240 may establish connections between the processing device 120 and the imaging device 110, the terminal(s) 140, and/or the storage device 130. The connection may be a wired connection, a wireless connection, any other communication connection that can enable data transmission and/or reception, and/or any combination of these connections. The wired connection may include, for example, an electrical cable, an optical cable, a telephone wire, or the like, or any combination thereof. The wireless connection may include, for example, a Bluetooth™ link, a Wi-Fi™ link, a WiMax™ link, a WLAN link, a ZigBee link, a mobile network link (e.g., 3G, 4G, 5G), or the like, or any combination thereof. In some embodiments, the communication port 240 may be and/or include a standardized communication port, such as RS232, RS485. In some embodiments, the communication port 240 may be a specially designed communication port. For example, the communication port 240 may be designed in accordance with the digital imaging and communications in medicine (DICOM) protocol.

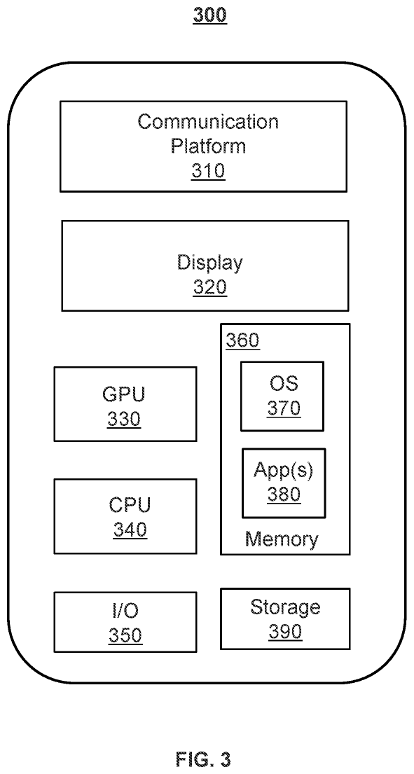

FIG. 3 is a schematic diagram illustrating exemplary hardware and/or software components of a mobile device 300 according to some embodiments of the present disclosure. In some embodiments, a terminal(s) 140 and/or a processing device 120 may be implemented on a mobile device 300, respectively.

As illustrated in FIG. 3, the mobile device 300 may include a communication platform 310, a display 320, a graphics processing unit (GPU) 330, a central processing unit (CPU) 340, an I/O 350, a memory 360, and a storage 390. In some embodiments, any other suitable component, including but not limited to a system bus or a controller (not shown), may also be included in the mobile device 300.

In some embodiments, the communication platform 310 may be configured to establish a connection between the mobile device 300 and other components of the imaging system 100, and enable data and/or signal to be transmitted between the mobile device 300 and other components of the imaging system 100. For example, the communication platform 310 may establish a wireless connection between the mobile device 300 and the imaging device 110, and/or the processing device 120. The wireless connection may include, for example, a Bluetooth™ link, a Wi-Fi™ link, a WiMax™ link, a WLAN link, a ZigBee link, a mobile network link (e.g., 3G, 4G, 5G), or the like, or any combination thereof. The communication platform 310 may also enable the data and/or signal between the mobile device 300 and other components of the imaging system 100. For example, the communication platform 310 may transmit data and/or signals inputted by a user to other components of the imaging system 100. The inputted data and/or signals may include a user instruction. As another example, the communication platform 310 may receive data and/or signals transmitted from the processing device 120. The received data and/or signals may include imaging data acquired by a detector of the imaging device 110.

In some embodiments, a mobile operating system (OS) 370 (e.g., iOS™ Android™, Windows Phone™, etc.) and one or more applications (App(s)) 380 may be loaded into the memory 360 from the storage 390 in order to be executed by the CPU 340. The applications 380 may include a browser or any other suitable mobile apps for receiving and rendering information relating to the imaging system 100 from the processing device 120. User interactions with the information stream may be achieved via the I/O 350 and provided to the processing device 120 and/or other components of the Imaging system 100 via the network 150.

To implement various modules, units, and their functionalities described in the present disclosure, computer hardware platforms may be used as the hardware platform(s) for one or more of the elements described herein. A computer with user interface elements may be used to implement a personal computer (PC) or another type of work station or terminal device, although a computer may also act as a server if appropriately programmed. It is believed that those skilled in the art are familiar with the structure, programming and general operation of such computer equipment and as a result the drawings should be self-explanatory.

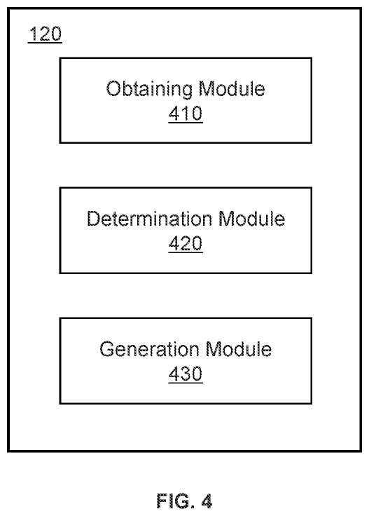

FIG. 4 is a block diagram illustrating an exemplary processing device according to some embodiments of the present disclosure. In some embodiments, the processing device 120 may include an obtaining module 410, a determination module 420, and a generation module 430.

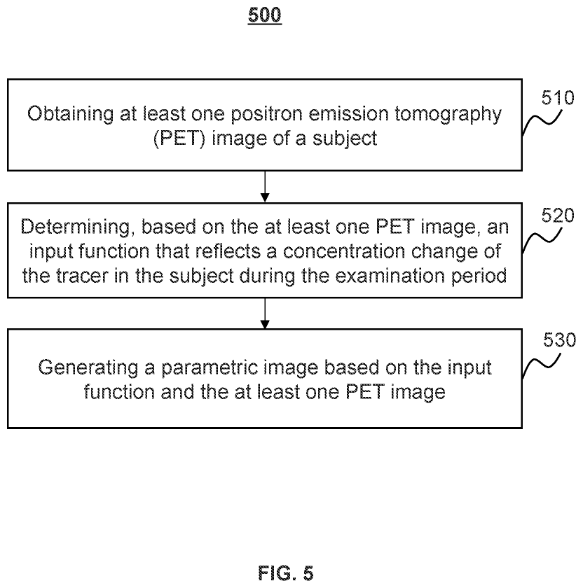

The obtaining module 410 may be configured to obtain data and/or information associated with the imaging system 100. The data and/or information associated with the imaging system 100 may include a PET image of a subject, an input function (e.g., a reference input function, a candidate input function) of the subject, a parametric image, a compartment model, a relationship function, or the like, or any combination thereof. For example, the obtaining module 410 may obtain at least one PET image of a subject. The at least one PET image may be obtained by performing a multi-point scan or a dual injection scan on the subject. More descriptions of the obtaining of the at least one PET image may be found elsewhere in the present disclosure (e.g., FIG. 5, 7A, 7B, and descriptions thereof). As another example, the obtaining module 410 may obtain a reference function of a subject. In some embodiments, the obtaining module 410 may obtain the data and/or the information associated with the imaging system 100 from one or more components (e.g., the terminal device 140, the storage device 130, the imaging device 110) of the imaging system 100 via the network 150.

The determination module 420 may be configured to determine an input function. The input function may reflect a concentration change of a tracer in a subject during an examination period. In some embodiments, the determination module 420 may determine an input function based on at least one PET image of a subject. For example, the determination module 420 may determine a candidate input function that reflects a concentration change of a tracer in a subject during a scan period based on a PET image corresponding to the scan period. As another example, the determination module 420 may determine an input function by transforming a reference input function based on a plurality of candidate input functions. More descriptions of the determination of the input function may be found elsewhere in the present disclosure (e.g., FIG. 5, 7A, 7B, and descriptions thereof).

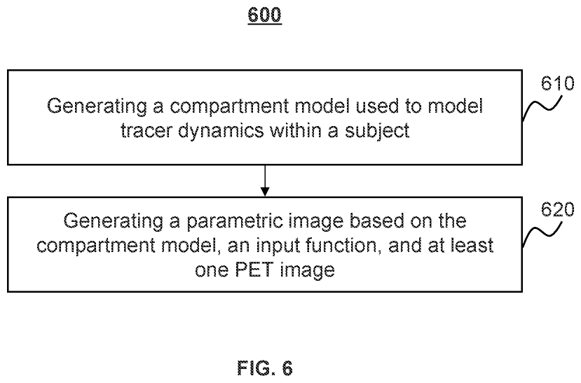

The generation module 430 may be configured to generate a parametric image based on an input function and at least one PET image. The parametric image may reflect a kinetic parameter of a tracer in a subject. In some embodiments, the generation module 430 may generate a compartment model used to model tracer dynamics within a subject. In some embodiments, the generation module 430 may generate a parametric image based on a compartment model, an input function, and at least one PET image. For example, the generation module 430 may generate a relationship function between a compartment model, an input function, and at least one PET image. The generation module 430 may generate a parametric image based on the relationship function according to a maximum likelihood estimation algorithm. More descriptions of the generation of the parametric image may be found elsewhere in the present disclosure (e.g., FIG. 5-6, and descriptions thereof).

It should be noted that the above description of the processing device 120 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more modules may be combined into a single module. For example, the determination module 420 and the generation module 430 may be combined into a single module, which may determine both the input function and the parametric image. In some embodiments, one or more modules may be added or omitted in the processing device 120. For example, the processing device 120 may further include a storage module (not shown in FIG. 4) configured to store data and/or information (e.g., the PET data, the input function, the parametric image) associated with the imaging system 100.

FIG. 5 is a flowchart illustrating an exemplary process for generating a parametric image according to some embodiments of the present disclosure. In some embodiments, the process 500 may be implemented in the imaging system 100 illustrated in FIG. 1. For example, the process 500 may be stored in the storage device 130 and/or the storage (e.g., the storage device 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3, one or more modules as illustrated in FIG. 4). The operations of the illustrated process presented below are intended to be illustrative. In some embodiments, the process 500 may be accomplished with one or more additional operations not described, and/or without one or more of the operations discussed. Additionally, the order in which the operations of the process 500 as illustrated in FIG. 5 and described below is not intended to be limiting.

In 510, the processing device 120 (e.g., the obtaining module 410) may obtain at least one PET image of a subject.

The at least one PET image may include a two-dimensional (2D) image, a 3D image, a 4D image (also referred to as a dynamic image) (e.g., a series of 3D images over time), and/or any related image data (e.g., scan data, projection data), or the like.

In some embodiments, the at least one PET image may be generated based on PET data acquired during an examination period in which the subject is injected with a tracer (also referred to as “PET tracer molecules” or “PET tracer”). The tracer may undergo positron emission decay and emit positrons. A positron has the same mass as and the opposite electrical charge to an electron, and it undergoes an annihilation (also referred to as an “annihilation event” or a “coincidence event”) with an electron (which may naturally exist in abundance within the subject) as the two particles collide. An electron-positron annihilation may result in two particles (e.g., two 511 keV gamma photons), which may travel in opposite directions with respect to one another. In a PET scan of the subject, particles produced by annihilation events may reach and be detected by detector units of a PET scanner. The detector units may acquire information regarding information (e.g., time information, trajectory information) of the particles (also referred to as the “PET data”). In some embodiments, the PET data may include list-mode data or sinogram data.

The distribution of the tracer may indicate information of biological activities in the subject. For example, one or more atoms of the tracer may be chemically incorporated into biologically active molecules in the subject. The active molecules may become concentrated in a tissue of interest within the subject. The tracer may include [¹⁵O]H₂O, [¹⁵O]butanol, [¹¹C]butanol, [¹⁸F]fluorodeoxyglucose (FDG), [⁶⁴Cu]diacetyl-bis (64Cu-ATSM), [¹⁸F]fluoride, 3′-deoxy-3′-[¹⁸F]fluorothymidine (FLT), [¹⁸F]-fluoromisonidazole (FMISO), gallium, thallium, or the like, or any combination thereof.

In some embodiments, the processing device 120 may obtain the PET data from one or more components (e.g., the imaging device 110, the terminal 140, and/or the storage device 130) of the imaging system 100 or an external storage device via the network 150. For example, the imaging device 110 may transmit acquired PET data (e.g., projection data) to a storage device (e.g., the storage device 130 or an external storage device) for storage. The processing device 120 may obtain the PET data from the storage device. As another example, the processing device 120 may obtain the PET data from the imaging device 110 directly. In some embodiments, the acquisition the PET data by the imaging device 110 and the transmission of the PET data to the processing device 120 may be performed substantially in real-time. Alternatively, the processing device 120 may obtain the PET data (e.g., from a storage device) after the PET data has been collected for a period.

After the PET data is obtained, the processing device 120 may generate the at least one PET image based on the PET data according to one or more image reconstruction algorithms. The at least one PET image may present the uptake of the tracer by the subject. Exemplary image reconstruction algorithms may include an iterative algorithm, an analytic algorithm, etc. The iterative algorithm may include a Maximum Likelihood Estimation (MLE) algorithm, an ordered subset expectation maximization (OSEM), a 3D reconstruction algorithm, etc. The analytic algorithm may include a filtered back projection (FBP) algorithm.

In some embodiments, the at least one PET image may include a plurality of PET images. The PET images may be obtained by performing a multi-point scan on the subject during the examination period. For example, all the tracer may be injected into the subject at an initial time point during the examination period. A plurality of PET scans may be sequentially performed on the subject during a plurality of scan periods after the initial time point. Each of the plurality of PET scans may be performed during one of the plurality of scan periods to acquire a set of PET data of the subject. The set of PET data acquired in a PET scan may be used to reconstruct one of the plurality of PET images. In some embodiments, a total time of the plurality of scan periods of the multi-point scan may be less than or equal to 10 minutes.

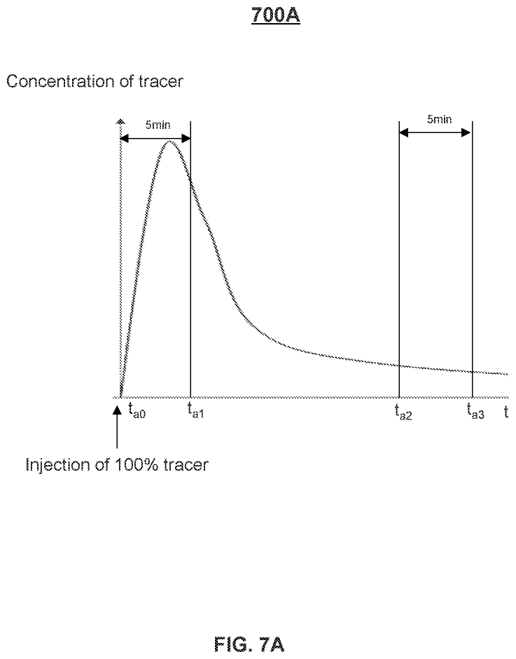

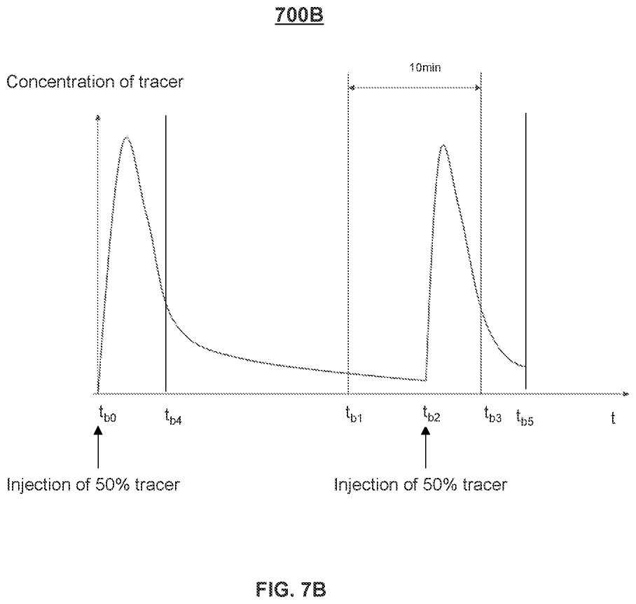

In some embodiments, a number (or count) of the PET scans in the multi-point scan, a duration of each PET scan, and/or a time interval between two adjacent PET scans may be manually set by a user of the imaging system 100, or determined by one or more components (e.g., the processing device 120) of the imaging system 100 according to different situations. In some embodiments, the duration of each PET scan may be the same or different. For example, each PET scan may last 5 minutes. More descriptions regarding a multi-point scan may be found elsewhere in the present disclosure. See, e.g., FIG. 7A and relevant descriptions thereof.

Alternatively, the at least one PET mage may include a single PET image. The single PET image may be obtained by performing a dual injection scan on the subject. For example, a first portion of the tracer may be injected into the subject at a first time point during the examination period, and a second portion of the tracer may be injected into the subject at a second time point after the first time point during the examination period. The first portion and the second portion may be the same or different. For example, a ratio of the first portion and the second portion may be equal to 0.8, 0.9, 1, 1.1, 1.2, or the like. A PET scan may be performed on the subject during a scan period, which may start after the first time point and before the second time point, and end after the second time point. In some embodiments, the scan period of the dual injection scan may be less than or equal to 10 minutes.

In some embodiments, a time interval between the first time point and the second time point, and/or a duration of the PET scan may be manually set by a user of the imaging system 100, or determined by one or more components (e.g., the processing device 120) of the imaging system 100 according to different situations. More descriptions regarding a multi-point scan may be found elsewhere in the present disclosure. See, e.g., FIG. 7B and relevant descriptions thereof.

In 520, the processing device 120 (e.g., the determination module 420) may determine, based on the at least one PET image, an input function that reflects a concentration change of the tracer in the subject during the examination period.

In some embodiments, the input function may be represented as a time activity curve (TAC) associated with the tracer. For example, the input function may be represented as a plasma TAC that indicates the concentration change of the tracer in the plasma, and/or a blood TAC that indicates the concentration change of the tracer in the blood.

In some embodiments, the input function determined in 520 may include a plurality of input functions corresponding to different portions of the subject. Merely by way of example, the processing device 140 may determine an input function for each physical point of the subject. A physical point of the subject refers to a portion of the subject that corresponds to an element (e.g., a pixel or a voxel) in the at least one PET image. The input functions corresponding to different portions of the subject may be different due to, for example, a dispersion effect, a time delay effect, etc. The dispersion effect and the time delay effect may be caused by blood circulation. Specifically, the dispersion effect may be caused by factors including, e.g., the inhomogeneous velocity of the blood in different blood vessels of the subject. The time delay effect may be caused by a distance between a blood sampling site (e.g., an injection position) and a specific organ or tissue in the subject. Because of the dispersion effect and the time delay effect, different portions of the subject may have different tracer concentrations at the same time, thereby having different input functions. More descriptions of the time delay effect may be found in, for example, Chinese Patent Application No. 201910383290.5 filed on Jun. 6, 2019 entitled “IMAGE RECONSTRUCTION METHOD, DEVICE, MEDICAL IMAGING EQUIPMENT AND STORAGE MEDIUM” the contents of which are hereby incorporated by reference.

In some embodiments, the input function (e.g., a plasma TAC) may be obtained using arterial samples technique, an image-derived input function technique, a population-based input function technique, venous blood sample scaling method or the like, or a combination thereof. Using the arterial samples technique, the arterial blood of the subject may be sampled to measure the input function of the subject. Using the image-derived input function technique, the input function of the subject may be determined based on one or more PET images (e.g., the at least one PET image determined in 510). For example, the processing device 120 may determine an ROI (e.g., a region associated with the heart or arterial blood) from each of the one or more PET images. The processing device 120 may determine a blood TAC based on the ROI identified from each PET image, and designate the blood TAC as the plasma TAC. The plasma TAC determined based on the one or more PET images may be also referred to as an image-derived input function. Using the population-based input function technique, the input function of the subject may be determined based on a plurality of sample input functions of a plurality of sample subjects (e.g., patients). The sample input functions may be determined based on the arterial sample technique. For example, the plurality of sample input functions of the plurality of sample subjects may be normalized and/or averaged to obtain the input function of the subject. Using the venous blood sample scaling technique, a venous sample of a certain time post injection may be extracted and used to determine the correct scaling factor for the population-based input function or the image-derived input function.

In some embodiments, a degree of similarity between each sample subject and the subject may be higher than a similarity threshold (e.g., 90%, 95%). The degree of similarity between a sample subject and the subject may be determined based on feature information of the sample subject and feature information of the subject. The feature information may include the gender, the age, a body shape (e.g., a thickness, a height, a width), a physiological state (e.g., a cardiac output), or any other features that may affect a metabolic rate of the tracer. For example, the processing device 120 may determine an average input function of the sample input functions of the sample subjects as a population-based input function of the subject. As another example, the processing device 120 may select a sample input function, the sample subject of which has the highest degree of similarity to the subject, among the plurality of sample input functions. The processing device 120 may then designate the selected sample input function as the population-based input function of the subject. As still another example, the processing device 120 may modify the selected sample input function (e.g., modify a shape of the selected sample input function) based on the feature information of the subject and the sample subject corresponding to the selected sample input function, for example, a cardiac output difference between the subject and the sample subject. The processing device 120 may further designate the modified sample input function as the population-based input function of the subject. Accordingly, by determining the population-based input function of the subject based on the feature information of the subject, the accuracy the population-based input function may be improved.

In some embodiments, the at least one PET image obtained in operation 510 may include a plurality of PET images acquired by a multi-point scan. More descriptions regarding the determination of the input function for the multi-point scan may be found elsewhere in the present disclosure. See, e.g., FIG. 7A and relevant descriptions thereof. Alternatively, the at least one PET image obtained in operation 510 may include a single PET image acquired by a dual injection scan. More descriptions regarding the determination of the input function for the dual injection scan may be found elsewhere in the present disclosure. See, e.g., FIG. 7B and relevant descriptions thereof.

In 530, the processing device 120 (e.g., the generation module 430) may generate a parametric image based on the input function and the at least one PET image.

In some embodiments, the parametric image may reflect a kinetic parameter of the tracer in the subject. As used herein, the term “kinetic parameter” refers to a physiological parameter associated with the kinetics of a tracer after the tracer is injected into a subject. For instance, the kinetic parameter may include a transportation rate of the tracer from a plasma to tissue (or referred to as a K₁ parameter of the tracer), a transportation rate of the tracer from the tissue to the plasma (or referred to as a K₂ parameter of the tracer), a concentration of plasma in the tissue, a perfusion rate of the tracer, a receptor binding potential of the tracer, a ki parameter of the tracer, or the like, or any combination thereof. The parametric image may aid the evaluation of the physiology (functionality) and/or anatomy (structure) of an organ and/or tissue in the subject.

In some embodiments, the parametric image may present a value of a kinetic parameter corresponding to one or more time points during the examination period. For example, the parametric image may include one or more static images corresponding to one or more time points. As another example, the parametric image may include a dynamic parametric image, such as a graphic interchange format (GIF) image that reflects the change of the kinetic parameter with respect to time.

In some embodiments, the processing device 120 may generate a compartment model (e.g., a two-tissue compartment model) used to model tracer dynamics within the subject. Further, the processing device 120 may generate the parametric image based on the compartment model, the input function, and the at least one PET image. More descriptions for the generation of the parametric image may be found elsewhere in the present disclosure (e.g., FIG. 6 and the descriptions thereof). In some embodiments, the processing device 120 may generate the parametric image based on the input function and the at least one PET image according to a non-linear parametric estimation algorithm. More descriptions regarding a non-linear parametric estimation algorithm may be found elsewhere in the present disclosure. See, e.g., operation 620 and relevant descriptions thereof.

According to some embodiments of the present disclosure, the at least one PET image may be obtained by performing a multi-point scan or a dual injection scan on the subject. The input function may then be generated based on an image-derived input function and a population-based input function. The parametric image (e.g., a Ki image) may further be generated based on the input function and the at least one PET image. Compared with the conventional approach (e.g., parametric imaging using a Patlak model), the systems and methods disclosed herein may be used to generate a parametric image with a relatively shorter imaging time (e.g., less than 10 minutes), which may improve the imaging efficiency and promote a clinical application of the parametric imaging.

It should be noted that the above description regarding process 500 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the process 500 may include an additional operation to transmit the determined parametric image to a terminal device (e.g., the terminal 140) for display. As another example, process 500 may include an additional operation to store information and/or data (e.g., the at least one PET image, the input function, the parametric image) in a storage device (e.g., the storage device 130) disclosed elsewhere in the present disclosure.

In some embodiments, the at least one PET image of the subject may include at least one gated PET image of the subject. Merely by way of example, the processing device 120 may gate the PET data of the subject acquired during the examination period. For example, the processing device 120 may gate the PET data into a plurality of groups. Different groups may correspond to different time periods or phases of a motion (e.g., a respiratory motion, a cardiac motion). For example, different groups may correspond to different respiratory phases of the subject. The processing device 120 may reconstruct a plurality of gated PET images using the plurality of groups of gated PET data. For illustration purposes, a first group of gated PET data may correspond to an end inspiration phase, and a second group of gated PET data may correspond to an end-expiration phase. The processing device 120 may reconstruct a first gated PET image using the first group of gated PET data and a second gated PET image using the second group of gated PET data.

FIG. 6 is a flowchart illustrating an exemplary process for generating a parametric image according to some embodiments of the present disclosure. In some embodiments, the process 600 may be implemented in the imaging system 100 illustrated in FIG. 1. For example, the process 600 may be stored in the storage device 130 and/or the storage (e.g., the storage device 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3, one or more modules as shown in FIG. 4). The operations of the illustrated process presented below are intended to be illustrative. In some embodiments, the process 600 may be accomplished with one or more additional operations not described, and/or without one or more of the operations discussed. Additionally, the order in which the operations of the process 600 as illustrated in FIG. 6 and described below is not intended to be limiting. In some embodiments, one or more operations of the process 600 may be performed to achieve at least part of operation 530 as described in connection with FIG. 5.

In 610, the processing device 120 (e.g., the generation module 430) may generate a compartment model used to model tracer dynamics within the subject.

In some embodiments, the compartment model may be a two-tissue compartment model. The two-tissue compartment model may include a first compartment model that describes the transport of the tracer between the blood/plasma and the tissue, and a second compartment model that describes a phosphorylation process of the tracer (e.g., FDG) in cells. For example, the first compartment model may be used to model a forward transport of the tracer from the plasma to the tissue and a backward transport of the tracer from the plasma to the tissue. The second compartment model may be used to model a phosphorylation process of the tracer in the tissue of the subject and a dephosphorylation process of the tracer in the tissue of the subject. As used herein, a phosphorylation process refers to a chemical addition process of a phosphoryl group (PO³⁻) to an organic molecule, and a dephosphorylation process refers to a removal process of the phosphoryl group. For example, phosphorylated FDG (e.g., FDG-6 phosphate) may be generated after phosphorylation of the FDG by hexokinase, and the phosphorylated FDG may be metabolically trapped and sequestered in cells of the tissue.

Merely by way example, the two-tissue compartment model may be represented using the following Equations (1) and (2):

$$\frac{{dC}_{1}}{dt} = {K}_{1} {C}_{p} + {k}_{4} {C}_{2} - {k}_{2} {C}_{1} - {k}_{3} {C}_{1} , \qquad ( 1 )$$

$$\frac{{dC}_{2}}{dt} = {k}_{3} {C}_{1} - {k}_{4} {C}_{2} , \qquad ( 2 )$$

where

$$\frac{{dC}_{1}}{dt}$$

denotes to a concentration change rate of the tracer in the first compartment (for some tracers it may be tissue) of the subject;

$$\frac{{dC}_{2}}{dt}$$

denotes a concentration change rate of the tracer in the second compartment (for some tracers it may be tracers cells, or phosphorylation process when using FDG as the tracer) of the subject; C₁ denotes a concentration of the tracer in the first compartment; C₂ denotes a concentration of the tracer in the second compartment; Cₚ denotes an input function (which may reflect the concentration of the tracer in the plasma); K₁ denotes a forward transport rate of the tracer from the plasma to the first compartment; k₂ denotes a backward transport rate of the tracer from the first compartment to the plasma; k₃ denotes a phosphorylation rate of the tracer (for FDG); k₄ denotes a dephosphorylation rate of the tracer (for FDG); and t denotes the time that has lapsed since the tracer is injected.

In some embodiments, the two-tissue compartment model may be constructed for each physical point of the subject to model the tracer dynamics at the physical point.

In 620, the processing device 120 (e.g., the generation module 430) may generate a parametric image based on the compartment model, the input function, and the at least one PET image.

In some embodiments, the processing device 120 may generate a relationship function between the compartment model, the input function, and the at least one PET image. Merely by way of example, assuming that most of the phosphorylated FDG is usually metabolically trapped and sequestered in cells without undergoing a dephosphorylation process (i.e., k₄=0), the relationship function between the compartment model, the input function, and the at least one PET image may be represented using the following Equation (3):

$$X ( t ) = {v}_{b} {C}_{p} + {C}_{1} + {C}_{2} = {v}_{b} {C}_{p} + \frac{{k}_{2} {K}_{1}}{{k}_{2} + {k}_{3}} exp ( - ( {k}_{2} + {k}_{3} ) t ) ⊗ {C}_{p} ( t ) + \frac{{k}_{3} {K}_{1}}{{k}_{2} + {k}_{3}} \int {C}_{p} dt , \qquad ( 3 )$$

where t denotes a time point, X(t) denotes a PET image corresponding to the time point t; v_{b} denotes a concentration of the plasma in the tissue; ⊗ denotes a convolution operation.

In some embodiments, the relationship function may be simplified according to the following Equation (4):

X(t)=v_{b}Cₚ+C₁+C₂=v_{b}Cₚ(t)K₁′exp(−k₂′t)⊗Cₚ(t)+KᵢCᵢ(t), (4)

where K′₁, k′₂, Kᵢ, and Cᵢ(t) may be represented according to Equation (5)-(8), respectively, as below:

$${K}_{1}^{′} = {K}_{1} \frac{{k}_{2} {K}_{1}}{{k}_{2} + {k}_{3}} , \qquad ( 5 )$$

$${k}_{2}^{′} = {k}_{2} + {k}_{3} , \qquad ( 6 )$$

$${K}_{i} = \frac{{k}_{3} {K}_{1}}{{k}_{2} + {k}_{3}} , \qquad ( 7 )$$

$${C}_{i} ( t ) = \int {C}_{p} dt , \qquad ( 8 )$$

Further, the processing device 120 may generate the parametric image based on the relationship function. In some embodiments, the processing device 120 may generate the parametric image based on the relationship function according to a non-linear parametric estimation algorithm. The non-linear parametric estimation algorithm refers to a non-linear algorithm used to determine the parametric image. The non-linear algorithm refers to an algorithm including one or more non-linear operations, such as a Logarithm operation, a square root operation, an exponential operation, an integral operation, or the like, or any combination thereof. For example, the non-linear algorithms may include an iterative algorithm. Exemplary iterative algorithms may include a maximum likelihood estimation algorithm (MLE) algorithm, a least square algorithm, an ordered subset expectation maximization (OSEM) algorithm, a maximum posterior probability (MAP) algorithm, a weighted least square (WLS) algorithm, or the like, or any combination thereof.

For example, Equation (5) may be constructed for each physical point of the subject. Merely by way of example, for a physical point, X(t) represents an element value (e.g., pixel or voxel values) that correspond to the physical point in each PET image, and Kᵢ represents the Kᵢ value of the physical point. The processing device 120 may determine the value of a kinetic parameter of the physical point based on the Equation (4) corresponding to the physical point. For illustration purposes, Kᵢ is taken as an exemplary kinetic parameter of the physical point to be determined. Assuming that element values of a physical point in different PET images approximately satisfy a Gaussian distribution, the Kᵢ of the physical point may be estimated using the least square algorithm. As another example, assuming that element values in the PET image approximately satisfy a Poisson distribution, the Kᵢ of the physical point may be estimated using the maximum likelihood estimation algorithm with the following Equations (9)-(12):

$${K}_{i}^{p + 1} = \frac{{K}_{i}^{p}}{{\sum }_{t} {C}_{i} ( t )} {\sum }_{t} \frac{{C}_{i} ( t ) X ( t )}{{\hat{X}}^{p} ( t )} , \qquad ( 9 )$$

$${v}_{b}^{p + 1} = \frac{{v}_{b}^{p}}{{\sum }_{t} {C}_{p} ( t - {t}_{d} )} {\sum }_{t} \frac{{C}_{p} ( t ) X ( t )}{{\hat{X}}^{p} ( t )} , \qquad ( 10 )$$

$${K}_{1}^{{′}^{p + 1}} = \frac{{K}_{1}^{{′}^{p}}}{{\sum }_{t} {e}^{- {k}_{2} {,}^{{p}_{t}}} ⊗ {C}_{p} ( t )} {\sum }_{t} \frac{{e}^{- {k}_{2}^{p} t} ⊗ {C}_{p} ( t ) X ( t )}{{\hat{X}}^{p} ( t )} , \qquad ( 11 )$$

$${k}_{2}^{′ p + 1} = {k}_{2}^{p} \frac{{\sum }_{t} {tK}_{1}^{{′}^{p}} ( exp ( - {k}_{2}^{′ p} t ) ) ⊗ {C}_{p} ( t )}{{\sum }_{t} \frac{{tK}_{1}^{{′}^{p}} ( exp ( - {k}_{2}^{′ p} t ) ) ⊗ {C}_{p} ( t ) X ( t )}{{\hat{X}}^{p} ( t )} ,} \qquad ( 12 )$$

where \hat{X}^{p}(t) denotes an element value of the physical point in an estimated PET image corresponding to the time point t; p denotes an iteration number; ⊗ denotes a convolution operation; and t_{d} denotes the time needed for the tracer to reach the physical point. The \hat{X}^{p}(t) may be determined according to Equation (13) as below:

\hat{X}^{p}(t)=v_{b}^{p}Cₚ+K′₁^{p}e^{-k′}^{₂}^{^{p}}^{t}⊗Cₚ(t)+Kᵢ^{p}Cᵢ(t), (13)

In Equations (9)-(12), the X(t) may have a known measured value, and the values of the parameters Kᵢ, v_{b}, K′₁, and k₂′ may be updated iteratively. In some embodiments, X(t)/\hat{X}^{p}(t) in Equations (9)-(12) may measure a difference between a measured element value and a predicted element value of the physical point. The update of the parameters Kᵢ, v_{b}, K′₁, and k₂′ may be performed based on X(t)/\hat{X}^{p} (t) so as to minimize the difference between the measured element value and the predicted value of the physical point.

In some embodiments, an alternate update approach may be used to estimate the values of the parameters Kᵢ, v_{b}, K′₁, and k₂′. For example, a plurality of iterations may be performed to update the parameters Kᵢ, v_{b}, K′₁, and k₂′. In each iteration of the plurality of iterations, Equations (9)-(12) may be sequentially performed to update the parameters Kᵢ, v_{b}, K′₁, and k₂′. The plurality of iterations may be performed to update the parameters until a termination condition is satisfied.

If the termination condition is satisfied in a current iteration, the processing device 120 may designate the Kᵢ obtained in the current iteration as the final Kᵢ of the physical point. For example, the termination condition may be satisfied if the difference between the Kᵢ obtained in the current iteration and a preset value is smaller than a threshold. As another example, the termination condition may be satisfied if the variation of the Kᵢ obtained in two or more consecutive iterations is smaller than a threshold (e.g., a constant). As still another example, the termination condition may be satisfied when a specified number (or count) of iterations has been performed.

In some embodiments, the processing device 120 may determine one or more kinetic parameters of each physical point of the subject, and generate at least one parametric image corresponding to the kinetic parameters. For example, the parametric image may include a Ki image that reflects a rate of the tracer transport from the plasma to the tissue of the subject. The Ki image may be used for the identification and/or evaluation of a tumor in the subject.

It should be noted that the above description regarding the process 600 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 7A is a schematic diagram illustrating an exemplary multi-point scan according to some embodiments of the present disclosure. As illustrated in FIG. 7A, the horizontal axis represents time tin an examination period 700A, and the vertical axis represents a concentration of a tracer in a subject during the examination period 700A. The examination period 700A lasts from an initial time point tₐ₀ to an end time point tₐ₃. The tracer is injected into the subject at the initial time point tₐ₀ in the examination period 700A. The multi-point scan may be performed on the subject to acquire the at least one PET image as described in connection with operation 510. The at least one PET image may include a first PET image and a second PET image of the subject. The first PET image may be acquired by performing a first PET scan on the subject during a first scan period from tₐ₀ to tₐ₁ The second PET image may be acquired by performing a second PET scan on the subject during a second scan period from tₐ₂ to tₐ₃. The duration of the first scan period from tₐ₀ to tₐ₁, and the duration of the second scan period from tₐ₂ to tₐ₃ may both be 5 minutes. Merely by way of example, if the examination period 700A lasts 60 minutes, and the tracer is injected into the subject at the 0th minute of the examination period 700A, the first scan period may last from the 0th minute to the 5th minute, and the second scan period may last from the 55th minute to the 60th minute of the examination period 700A. The total time of the first scan period and the second scan period may be 10 minutes.

In some embodiments, the processing device 120 may determine an input function 11 that reflects a concentration change of the tracer in the subject during the examination period 700A. For example, for each of the plurality of scan periods of the multi-point scan, the processing device 120 may determine a candidate input function (also referred to as an image-derived input function) based on the PET image corresponding to the scan period. The candidate input function may reflect a concentration change of the tracer in the subject during the scan period. The processing device 120 may obtain a reference input function relating to the subject. The reference input function may reflect a predicted concentration change of the tracer in the subject during a period other than the scan periods in the examination period 700A. For example, the reference input function may be a population-based input function relating to the subject as aforementioned. Further, the processing device 120 may generate the input function by transforming the reference input function based on the plurality of candidate input functions. For example, the processing device 120 may modify (e.g., scale) the reference input function such that an end of the modified reference input function is consistent with (e.g., coincides with) a corresponding end of a candidate input function. An end of the modified reference input function and an end of a candidate input function may be regarded as being corresponding to each other if they correspond to a same (or substantially same) time point. The processing device 120 may generate the input function 11 by combining the modified reference input function and the plurality of candidate input functions.

Merely by way of example, referring to FIG. 7A, the processing device 120 may determine a candidate input function F1 that reflects a concentration change of the tracer in the subject during the first scan period from tₐ₀ to tₐ₁ based on the first PET image. The processing device 120 may determine a candidate input function F2 that reflects a concentration change of the tracer in the subject during the second scan period from tₐ₂ to tₐ₃ based on the second PET image. The processing device 120 may obtain a reference input function R1 that reflects a predicted concentration change of the tracer in the subject during a period from tₐ₁ to tₐ₂. The processing device 120 may scale the reference input function R1 such that a value of the scaled reference input function at the time point tₐ₁ is equal to a value of the candidate input function F1 at the time point tₐ₁, and a value of the scaled reference input function at the time point tₐₑ is equal to a value of the candidate input function F2 at the time point tₐₑ. The processing device 120 may determine the input function 11 corresponding to the examination period 700A from tₐ₀ to tₐ₃ by combining the candidate input function F1 corresponding to the first scan period from tₐ₀ to tₐ₁, the scaled reference function corresponding to the time period from tₐ₁ to tₐ₂, and the candidate input function F2 corresponding to the second scan period from tₐ₂ to tₐ₃.

For illustration purposes, the input function 11 may be represented according to Equation (14):

$${C}_{p} ( t ) {=}^{△} \{ \begin{matrix}{C}_{image} ( t ) & {t}_{a 0} \leq t \leq {t}_{a 1} and {t}_{a 2} \leq t \leq {t}_{a 3} \\ μ {e}^{- γ ( t - {t}_{a 1} )} {C}_{p 0} ( t ) & {t}_{a 1} \leq t \leq {t}_{a 2}\end{matrix} , \qquad ( 14 )$$

where t denotes a time point in the examination period 700A; Cₚ(t) denotes the value of the input function 11 at the time point t; C_{image}(t) denotes the value of the candidate input function F1 or the candidate input function F2 at the time point t; Cₚ₀(t) denotes the reference input function R1; and γ and μ denote scaling constants to satisfy μCₚ₀(tₐ₁)=C_{image}(tₐ₁), and μe^{-γ(t}^{ₐ₂}^{-t}^{ₐ₁}⁾Cₚ₀(tₐ₂)=C_{image}(tₐ₂).

In some embodiments, the processing device 120 may perform an image registration operation on the first and second PET images before they are used to determine the input function. For example, the processing device 120 may register the first and second PET images based on image feature(s) of the first and second PET images according to one or more image registration algorithms. The image feature(s) may include a grayscale feature, a gradient feature, an edge feature, a texture feature, or the like, or any combination thereof. Exemplary image registration algorithms may include an intensity-based algorithm, a feature-based algorithm, a transformation model algorithm (e.g., a linear transformation model, a non-rigid transformation model), a spatial domain algorithm, a frequency domain algorithm, a single-modality algorithm, a multi-modality algorithm, an automatic algorithm, and an interactive algorithm, or the like, or any combination thereof. After the image registration operation is performed on the first and second images, a same position in each registered PET image may correspond to a same physical (or spatial) point of the subject.

It should be noted that the example illustrated in FIG. 7A is merely provided for the purposes of illustration, and not intended to limit the scope of the present disclosure. For persons having ordinary skills in the art, multiple variations and modifications may be made under the teachings of the present disclosure. Merely by way of example, the at least one PET image may further include a third PET image, and the multi-point scan may further include a third PET scan. The first scan period may last from the 0th minute to the 3th minute of the examination period 700A. The second scan period may last from the 30th minute to the 33th minute of the examination period 700A. The third PET image may be generated by performing the third PET scan on the subject during a third scan period, which lasts from the 57th minute to the 60th minute of the examination period 700A.

FIG. 7B is a schematic diagram illustrating an exemplary dual injection scan according to some embodiments of the present disclosure. As illustrated in FIG. 7B, the horizontal axis represents time tin an examination period 700B, and the vertical axis represents a concentration of a tracer in a subject during the examination period 700B. The examination period 700B lasts from a time point t_{b0} to an end time point t_{b3}. A first portion of the tracer (e.g., 50% tracer) is injected into the subject at the initial time point t_{b0} (i.e., a first injection is performed at t_{b0}), and a second portion of the tracer (e.g., 50% tracer) is injected into the subject at a time point t_{b2} after the time point t_{b0} (i.e., a second injection is performed at t_{b2}). The dual injection scan may be performed on the subject to acquire the at least one PET image as described in connection with operation 510. The at least one PET image may include a single PET image of the subject. The PET image may be acquired by performing a PET scan on the subject during a scan period from t_{b1} to t_{b3}. The time point t_{b1} is after the time point t_{b0} and before the time point t_{b2}. The time point t_{b3} is after the time point t_{b2}. The duration of the scan period from t_{b1} to t_{b3} may be, for example, 10 minutes. Merely by way of example, if the examination period 700B lasts 60 minutes, 50% tracer is injected into the subject at the 0th minute, 50% tracer is injected into the subject at the 55th minute, and the scan period may last from the 50th minute to the 60th minute of the examination period 700B.

In some embodiments, the processing device 120 may determine an input function 12 that reflects a concentration change of the tracer in the subject during the examination period 700B. For example, referring to FIG. 7B, based on the PET image, a candidate input function F3 (or referred to as a first candidate input function) that reflects a concentration change of the tracer in the subject during the scan period t_{b1} to t_{b3} may be determined. The processing device 120 may then determine a candidate input function F4 (or referred to as a second candidate input function) that reflects a concentration change of the tracer in the subject during a first period after the time point t_{b0} based on the candidate input function F3, the first portion, and the second portion.

Assuming that a ratio of the shape of the input function from the first injection and the shape of the input function from the second injection relates to a ratio of the first portion and the second portion, the processing device 120 may determine the candidate input function F4 based on the candidate input function F3, the first portion, and the second portion. For example, if the ratio of the first portion and the second portion is 1, the shape of the input function from the second injection may be the same as the shape of the input function from the first injection. As used herein, “an input function from an injection” refers to an input function corresponding to an early period after (e.g., immediately after) a tracer is injected into a subject. For example, the input function from the first injection may correspond to a period from t_{b0} to t_{b4} as illustrated in FIG. 7B, and the input function from the second injection may correspond to a period from t_{b2} to t_{b3} as illustrated in FIG. 7B.

The processing device 120 may obtain a reference input function R2 relating to the subject. The reference input function R2 may reflect a concentration change of the tracer in the subject during a second period from t_{b4} to t_{b1}. Further, the processing device 120 may generate the input function 12 by transforming the reference input function R2 based on the plurality of candidate input functions. For example, the processing device 120 may modify (e.g., scale) the reference input function R2 such that an end of the modified reference input function is consistent with (e.g., coincides with) a corresponding end of a candidate input function. The processing device 120 may generate the input function 12 by combining the modified reference input function and the plurality of candidate input functions.

Merely by way of example, referring to FIG. 7B, the processing device 120 may scale the reference input function R2 such that a value of the scaled reference input function at the time point t_{b4} is equal to a value of the candidate input function F4 at the time point t_{b4}, and a value of the scaled reference input function at the time point t_{b1} is equal to a value of the candidate input function F3 at the time point t_{b1}. The processing device 120 may determine the input function 12 corresponding to the examination period 700B from t_{b1} to t_{b3} by combining the candidate input function F4 corresponding to the period from t_{b0} to t_{b4}, the scaled reference function corresponding to the period from t_{b4} to t_{b1}, and the candidate input function F3 corresponding to the scan period from t_{b1} to t_{b3}.

It should be noted that the example illustrated in FIG. 7B is merely provided for the purposes of illustration, and not intended to limit the scope of the present disclosure. For persons having ordinary skills in the art, multiple variations and modifications may be made under the teachings of the present disclosure. For example, the tracer may be injected into the subject via three or more injections and two or more PET scans may be performed on the subject.

In some embodiments, the processing device 120 may determine a candidate input function F5 that reflects a concentration change of the tracer in the subject during a third period from t_{b3} to t_{b5} after the scan period. The time point t_{b5} is after the time point t_{b3}. The processing device 120 may determine the candidate input function F5 based on the first portion, the second portion, and the reference input function R2 corresponding to the second period from t_{b4} to t_{b1}. In some embodiments, the processing device 120 may determine the candidate input function F5 by scaling the reference input function R2 based on the ratio of the first portion and the second portion. For example, the processing device 120 may scale the reference input function R2 (or a portion of the reference input function R2 corresponding to a period after t_{b1} having the same duration as the third period) based on the ratio of the first portion and the second portion, and translate a scaled reference input function R2 to generate the candidate input function F5.

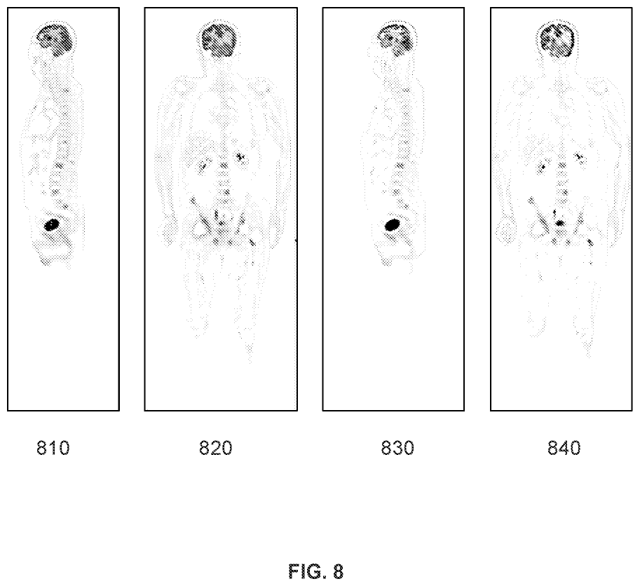

FIG. 8 illustrates exemplary Ki images of a patient according to some embodiments of the present disclosure. The Ki images include a Ki image 810 and a Ki image 820, which were generated based on at least one first PET image of the patient according to process 500 and process 600 of the present disclosure. A Ki image 830 and a Ki image 840 were generated based on at least one second PET image with a scan time of 40 minutes using a Patlak model. The at least one first PET image was acquired by performing a dual injection scan with 10 minutes, and the at least one second PET image was acquired by performing a continuous PET scan with 40 minutes. The Ki image 810 and the Ki image 830 correspond to a sagittal plane of the patient. The Ki image 820 and the Ki image 840 correspond to a coronal plane of the patient. It can be seen that the Ki image 810 has a similar resolution to the Ki image 830, and the Ki image 820 has a similar resolution to the Ki image 840. Accordingly, the systems and methods disclosed herein may be used to generate parametric images with a desired quality and accuracy with a relatively shorter imaging time (e.g., less than 10 minutes).

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

What is claimed is:

1. A method implemented on a computing device having at least one processor and at least one storage device, the method comprising:
obtaining at least one positron emission tomography (PET) image of a subject, wherein the at least one PET image is generated based on PET data acquired during an examination period, wherein in the examination period, the subject is injected with a tracer;
determining, based on the at least one PET image, an input function that reflects a concentration change of the tracer in the subject during the examination period; and
generating a parametric image based on the input function and the at least one PET image according to a non-linear parametric estimation algorithm, wherein the parametric image reflects a kinetic parameter of the tracer in the subject.

2. The method of claim 1, wherein the at least one PET image includes a plurality of PET images, and the obtaining at least one PET image of a subject comprises:
obtaining the plurality of PET images by performing a multi-point scan on the subject, wherein to perform the multi-point scan,
the tracer is injected into the subject at an initial time point during the examination period, and
a plurality of PET scans are performed on the subject during a plurality of scan periods after the initial time point, each of the plurality of PET scans being performed during one of the plurality of scan periods with a time interval between each pair of adjacent PET scans among the plurality of PET scans.

3. The method of claim 2, wherein the determining, based on the at least one PET image, an input function that reflects a concentration change of the tracer in the subject during the examination period comprises:
obtaining a reference input function relating to the subject;
for each of the plurality of scan periods, determining a candidate input function that reflects a concentration change of the tracer in the subject during the scan period based on the PET image corresponding to the scan period; and
generating the input function by transforming the reference input function based on the plurality of candidate input functions.

4. The method of claim 1, wherein the at least one PET image includes one PET image of the subject, and the obtaining at least one PET image of a subject comprises:
obtaining the PET image by performing a dual injection scan on the subject, wherein to perform the dual injection scan on the subject,
a first portion of the tracer is injected into the subject at a first time point during the examination period and a second portion of the tracer is injected into the subject at a second time point after the first time point during the examination period, and
a PET scan is performed during a scan period, the scan period starting after the first time point and before the second time point, the scan period ending after the second time point.

5. The method of claim 4, wherein the determining, based on the at least one PET image, an input function that reflects a concentration change of the tracer in the subject during the examination period comprises:
obtaining a reference input function relating to the subject;
determining, based on the PET image, a first candidate input function that reflects a concentration change of the tracer in the subject during the scan period;
determining a second candidate input function that reflects a concentration change of the tracer in the subject during a period after the first time point based on the first candidate input function, the first portion, and the second portion; and
generating the input function by transforming the reference input function based on the first and second candidate input functions.

6. The method of claim 1, wherein the generating a parametric image based on the input function and the at least one PET image according to a non-linear parametric estimation algorithm comprises:
generating a compartment model used to model tracer dynamics within the subject; and
generating the parametric image based on the compartment model, the input function, and the at least one PET image according to the non-linear parametric estimation algorithm.

7. The method of claim 6, wherein the compartment model is used to model at least one of:
a forward transport of the tracer from the plasma of the subject to the tissue of the subject,
a backward transport of the tracer from the plasma to the tissue,
a phosphorylation process in the tissue of the subject, or
a dephosphorylation process in the tissue of the subject.

8. The method of claim 6, wherein the generating the parametric image based on the compartment model, the input function, and the at least one PET image according to the non-linear parametric estimation algorithm comprises:
generating a relationship function between the compartment model, the input function, and the at least one PET image; and
generating the parametric image based on the relationship function according to the non-linear parametric estimation algorithm.

9. The method of claim 1, wherein the non-linear parametric estimation algorithm includes a maximum likelihood estimation (MLE) algorithm.

10. The method of claim 1, wherein the tracer is an ¹⁸F-fluorodeoxyglucose (FDG).

11. The method of claim 1, wherein the parametric image includes a Ki image.

12. A system, comprising:
at least one storage device storing executable instructions, and
at least one processor in communication with the at least one storage device, wherein when executing the executable instructions, the at least one processor causes the system to perform operations including:
obtaining at least one positron emission tomography (PET) image of a subject, wherein the at least one PET image is generated based on PET data acquired during an examination period, wherein in the examination period, the subject is injected with a tracer;
determining, based on the at least one PET image, an input function that reflects a concentration change of the tracer in the subject during the examination period; and
generating a parametric image based on the input function and the at least one PET image according to a non-linear parametric estimation algorithm, wherein the parametric image reflects a kinetic parameter of the tracer in the subject.

13. The system of claim 12, wherein the at least one PET image includes a plurality of PET images, and the obtaining at least one PET image of a subject comprises:
obtaining the plurality of PET images by performing a multi-point scan on the subject, wherein to perform the multi-point scan,
the tracer is injected into the subject at an initial time point during the examination period, and
a plurality of PET scans are performed on the subject during a plurality of scan periods after the initial time point, each of the plurality of PET scans being performed during one of the plurality of scan periods with a time interval between each pair of adjacent PET scans among the plurality of PET scans.

14. The system of claim 13, wherein the determining, based on the at least one PET image, an input function that reflects a concentration change of the tracer in the subject during the examination period comprises:
obtaining a reference input function relating to the subject;
for each of the plurality of scan periods, determining a candidate input function that reflects a concentration change of the tracer in the subject during the scan period based on the PET image corresponding to the scan period; and
generating the input function by transforming the reference input function based on the plurality of candidate input functions.

15. A method implemented on a computing device having at least one processor and at least one storage device, the method comprising:
obtaining at least one positron emission tomography (PET) image of a subject, wherein the at least one PET image is generated based on PET data acquired during an examination period, wherein in the examination period, the subject is injected with a tracer, a multi-point scan or a dual injection scan is performed on the subject, and a total time of one or more scan periods of the multi-point scan or the dual injection scan is less than or equal to 10 minutes; and
generating a parametric image based on the at least one PET image according to a non-linear parametric estimation algorithm, wherein the parametric image reflects a kinetic parameter of the tracer in the subject.

16. The method of claim 15, wherein the at least one PET image includes a plurality of PET images, and the obtaining at least one PET image of a subject comprises:
obtaining the plurality of PET images by performing the multi-point scan on the subject, wherein to perform the multi-point scan,
the tracer is injected into the subject at an initial time point during the examination period, and
a plurality of PET scans are sequentially performed on the subject during a plurality of scan periods after the initial time point, each of the plurality of PET scans being performed during one of the plurality of scan periods with a time interval between each pair of adjacent PET scans among the plurality of PET scans.

17. The method of claim 16, further comprising:
registering the plurality of PET images.

18. The method of claim 15, wherein the at least one PET image includes one PET image of the subject, and the obtaining at least one PET image of a subject comprises:
obtaining the PET image by performing the dual injection scan on the subject, wherein to perform the dual injection scan on the subject,
a first portion of the tracer is injected into the subject at a first time point during the examination period and a second portion of the tracer is injected into the subject at a second time point after the first time point during the examination period, and
a PET scan is performed during a scan period, the scan period starting after the first time point and before the second time point, the scan period ending after the second time point.

19. The method of claim 15, further comprising:
determining, based on the at least one PET image, an input function that reflects a concentration change of the tracer in the subject during the examination period.

20. The method of claim 15, wherein the parametric image includes a Ki image.
